(19) United States
(12) Patent Application Publication
(10) Pub. No.: US 2021/0016058 A1
(43) Pub. Date: Jan. 21, 2021

(54) INDWELLING VENOUS CANNULA

(71) Applicant: Jens EBNET, Schwerin (DE)

(72) Inventor: Jens EBNET, Schwerin (DE)

(21) Appl. No.: 17/040,342

(22) PCT Filed: Mar. 21, 2019
(86) PCT No.: PCT/EP2019/057097

§ 371 Date: Sep. 22, 2020

(30) Foreign Application Priority Data
Mar. 23, 2018 (DE) 20 2018 101 646.6

(51) Int. Cl. A61M 25/00; A61M 25/06; A61M 29/00; A61M 25/04; A61L 29/08; A61L 29/14

(52) U.S. Cl. CPC A61M 25/0017; A61M 25/0045; A61M 25/0606; A61M 29/00; A61M 5/142; A61M 25/0618; A61L 29/085; A61L 29/14; A61M 25/04

(57) ABSTRACT

The invention relates to an indwelling venous cannula for application to a living being, comprising a venous catheter (2), wherein a puncture needle (3) can be guided in a longitudinally displaceable manner in the venous catheter (2), wherein at least the part of the venous catheter (2), which is designed to remain in the living being, is formed from a puncture-resistant material or is coated with such a puncture-resistant material over its entire length or most of its length, in which the puncture needle (3) can be guided in a longitudinally displaceable manner.

The invention relates to an indwelling venous cannula for application to a living being, comprising a venous catheter, wherein a puncture needle can be guided in a longitudinally displaceable manner into the venous catheter.

DE 40 41 720 A1 describes an indwelling venous cannula comprising a venous catheter and a puncture needle displaceable in the latter, wherein the front part of the venous catheter penetrating into the body is made of stainless steel, and the rear part is made of a flexible material, for example a plastic.

Such indwelling venous cannulas have the disadvantage that the flexible part of the venous catheter can be very easily pierced through by contact with the puncture needle, and the indwelling venous cannula therefore has to be discarded. Moreover, there is the danger of the flexible part of the venous catheter or a part thereof being sheared off by contact with the puncture needle and remaining unnoticed in the living being and thus posing a danger to the latter.

The object of the invention is therefore to make available an improved indwelling venous cannula. The object of the invention is also to make available an improved puncture system in the sense of a general puncture system. In principle, with the improved indwelling venous cannula, it is in fact equally possible to advantageously puncture all body cavities and interstices, and all anatomical and pathological structures that need to be punctured, and to insert a catheter into them.

Nonetheless, the term “indwelling venous cannula” will be used below, even though it has a broader meaning in the sense of a general puncture system with which it is not just veins that can be punctured. The term “veins” as used below therefore also comprises in principle all body cavities and interstices and all anatomical and pathological structures that need to be punctured and provided with a catheter.

Thus, with the indwelling venous cannula according to the invention, it is additionally possible to puncture the trachea, pleural space, abdominal cavity, intestine, renal pelvis, urinary bladder and bones, for example. In addition, pathological structures such as abscesses in and on the living being can be punctured.

The indwelling venous cannula can either be removed directly again after the puncturing procedure or also left in place for a time. Advantageously, arterial blood vessels can also be punctured. Advantageously, sclerosed arterial blood vessels in particular can also be punctured, since the new puncture system is more stable than conventional indwelling venous cannulas and thus also permits a controlled advance, if appropriate an ultrasonically controlled advance, counter to a resistance.

The object is achieved with an indwelling venous cannula having the features of claim 1. Advantageous embodiments are described in the dependent claims.

It is proposed that at least the part of the venous catheter designed to remain in the living being is formed from a puncture-resistant material, or is coated with such a puncture-resistant material, over its entire length or most of its length in which the puncture needle can be guided in a longitudinally displaceable manner. The puncture resistance of the material relates to a possible piercing of the venous catheter by the puncture needle, which is intended to be prevented by the puncture-resistant material. The puncture-resistant material can be a metal, also in the form of a metal alloy, or a correspondingly puncture-resistant synthetic material or natural material. Examples of synthetic materials include carbon-fiber-reinforced laminate materials, polymers and/or Teflon, also in combination with one another. It is advantageous here if the venous catheter is not rendered too stiff by the puncture-resistant material, i.e. still has a certain flexibility. Moreover, with such materials, the venous catheter can be made compatible with MRT and nuclear spin.

The puncture-resistant material may no longer be present in particular in the (distal) end of the venous catheter in direct proximity to the patient. The thickness of the puncture-resistant material and/or the material density does not have to be constant along the entire lengthwise extent over which the puncture-resistant material is present on the venous catheter, and instead it can vary along the length. The puncture-resistant material does not have to be present all over. Gaps can deliberately be left to create, for example, a kind of predetermined “buckling point”.

The indwelling venous cannula according to the invention can be used in all sectors of medicine, i.e. not just in veins, but also in arteries or other anatomical regions of the human or animal body. Moreover, the indwelling venous cannula according to the invention is also intended to permit advantageous puncturing of technical systems and containers, for example the reservoirs of pumps inside and outside the human or animal body. It is conceivable, for example, to use it to puncture the reservoirs of insulin pumps and pain pumps in order to fill these up with an active substance. It can also conceivably be used to puncture medical port systems. For this purpose, the tip of the puncture needle can also be differently configured if appropriate, e.g. it can have a modified grinding. The advantage is that the puncture needle is always applied just briefly in the puncturing procedure controlled by the user, the venous catheter is then advanced over the puncture needle, and the puncture needle can then be directly retracted in a controlled manner. The further steps of the intended use can then be safely performed via the inserted, non-pointed venous catheter. By virtue of the novel properties of the indwelling venous cannula according to the invention, the venous catheter does not buckle for example when it comes to lie in a reservoir. In analogy to what has just been described, areas of use outside of medicine are also conceivable. For example, if reservoirs are to be filled up with fluids, vapors or gases, the reservoir has to be pierced, but a puncture needle then has to be immediately retracted again in order to avoid damage to the reservoir, and only a non-pointed catheter is intended to remain in the reservoir.

The invention has the advantage that the indwelling venous cannula is designed to be resistant to piercing, buckling and cutting. The danger of damage to the venous catheter is thereby minimized. After an inaccurate puncturing procedure, the indwelling venous cannula can thus be used again on the same living being under permanently sterile conditions since, even upon repeated displacement of the puncture needle relative to the venous catheter in the longitudinal direction, the latter cannot be damaged by the patient-side tip of the puncture needle. This constitutes a problem in conventional indwelling venous cannulas. In particular, even with slight buckling of the flexible venous catheter during use, the patient-side tip of the puncture needle, particularly upon repeated displacement of the puncture needle relative to the venous catheter in the longitudinal direction, can cause shearing off of the venous catheter or parts thereof. This disadvantage of conventional indwelling cannulas is overcome by the present invention. This improves patient safety.

Moreover, patient comfort is improved, since in particular the incision through the skin is painful and, with the indwelling venous cannula according to the invention, it is possible to actively and repeatedly seek out a blood vessel after an incision has been made through the skin and it is possible for the patient-side parts of the indwelling venous cannula, in particular the patient-side sharp end of the puncture needle, to remain permanently below the level of the skin in the living being. This reduces the number of incisions that need to be made in the skin in order to successfully position an indwelling venous cannula. This reduces the trauma to the surrounding tissue. Moreover, by reducing the number of incisions made in the skin, the number of possible points of entry for potential pathogens, such as bacteria and viruses, through the skin is also reduced.

Moreover, by virtue of the design and the improved material properties of the indwelling venous cannula according to the invention, accidental dislocation of the latter from the target structure is made difficult. On account of the more stable and more robust design, the indwelling venous cannula according to the invention is in particular also suitable for use in pressure infusions.

Further advantages of the invention are:
The indwelling venous cannula according to the invention is associated with reduced pain when the venous catheter is left in place in the patient, since the indwelling venous cannula according to the invention can adapt better to the anatomical structures. Irritation and inflammation of the veins can be prevented, since the indwelling venous cannula according to the invention causes considerably less internal trauma to the vein or another blood vessel, i.e. the vein wall is less irritated.
The danger of perforation during the advance of the venous catheter is reduced, even in sinuous and/or sclerosed blood vessels. This is of particularly great advantage in fragile blood vessels, in elderly patients and in special risk groups.
The indwelling venous cannula according to the invention makes it possible to vary the firmness of the venous catheter located in the blood vessel, since movements of the components are possible in all directions relative to one another.
The indwelling venous cannula according to the invention helps to avoid inaccurate puncture procedures and thus to reduce the danger of infection.
The puncture-resistant material also provides enhanced safety against buckling of the venous catheter.
On account of the venous catheter being strengthened by the puncture-resistant material, it is also possible to better counteract a possible blockage of the venous catheter. Blood can be obtained more easily and more permanently, even repeatedly. Moreover, it is possible to counter damage to corpuscular blood constituents and hemolysis, since the turbulent flow of the blood in the venous catheter is reduced by virtue of the buckling resistance of the latter. By comparison with existing venous catheters, the venous catheter overall is intended to have much better flow properties.

The puncture-resistant material can be formed, for example, by a tubular metal body or a metal coating of a base material of the venous catheter, which can be a plastic material, for example. The metal can be, for example, steel, e.g. stainless steel, or titanium or an alloy formed therewith or a bimetal. In an advantageous embodiment of the invention, the puncture-resistant material is arranged on the inner face of the venous catheter, i.e. the puncture-resistant material forms the inner wall of the venous catheter. A protective layer of the venous catheter, surrounding the puncture-resistant material from the outside, can be formed from plastic, for example.

In an alternative embodiment, the puncture-resistant material is arranged on the outer face of the venous catheter. However, it is also conceivable that the puncture-resistant material is arranged both on the inner face and on the outer face of the venous catheter. The puncture-resistant material can thus form either the inner or outer wall of the venous catheter or both walls. In this way, the improved indwelling venous cannula is more stable and more robust than previously known indwelling venous cannulas, while at the same time affording improved flexibility of the venous catheter. The puncture-resistant material is advantageously a material that has non-thrombogenic properties. Alternatively or additionally, the puncture-resistant material can be coated with an additional surface that has non-thrombogenic properties. This material property is intended to prevent the formation of thrombi (blood clots) on the venous catheter. The puncture-resistant material can be designed as a metal layer or also as a metal alloy (alloy). Advantageously, the puncture-resistant material is moreover compatible with magnetic resonance tomography (MRT), such that the inserted venous catheter or the components of the indwelling venous cannula lying in the body can also remain in the body in an MRT examination and do not damage the body during the MRT examination and cannot dislocate it in the magnetic field.

All of the aforementioned layers of the indwelling venous cannula advantageously have non-thrombogenic properties. Moreover, they advantageously have hypoallergenic and/or antimicrobial properties. Moreover, all of the layers can advantageously be made of highly slidable material or can be coated with such a material. Refinement of the layers or surfaces by means of nanotechnology methods is explicitly possible.

According to an advantageous development of the invention, the part of the venous catheter designed to remain outside the living being is also formed from a puncture-resistant material, or is coated with such a puncture-resistant material, over its entire length or most of its length. In this case too, the puncture-resistant material can be located both on the inner face and on the outer face or in each case both on the inner face and on the outer face. This can be the same puncture-resistant material as the puncture-resistant material mentioned in claim 1 or another puncture-resistant material. In this way, the puncture resistance can also be continued into the parts of the indwelling venous cannula arranged outside the body of the patient. Moreover, buckling resistance of parts of the indwelling venous cannula arranged outside the body is also afforded in this way.

In an advantageous development, the indwelling venous cannula is a peripheral indwelling venous cannula. It is moreover advantageous if the venous catheter of the peripheral indwelling venous cannula has a piercing length of at least 10 mm, e.g. 19 mm, and at most 200 mm, e.g. 50 mm. However, the venous catheter of the indwelling venous cannula can also have piercing lengths of up to 80, 100, 120 or 600 mm. The puncture needle and the indwelling venous cannula as a whole are then much longer. The piercing length of the venous catheter is the length corresponding to the length of the venous catheter lying in the living being. A living being is to be understood as meaning human and also animal living beings. An indwelling venous cannula according to the invention can be used in particular in the peripheral region, since the piercing length is short such that a venous catheter made of the puncture-resistant material, or a venous catheter made with a coating of puncture-resistant material, has no limitations during or after the application to a living being in relation to a conventional indwelling venous cannula and at the same time affords protection against piercing and cutting of the venous catheter.

The range of applications of the indwelling venous cannula according to the invention extends to all current indwelling venous cannulas in human medicine, including pediatric indwelling venous cannulas, and those in veterinary medicine. In particular, however, the indwelling venous cannula according to the invention is also intended to permit indwelling venous cannulas having greater diameters than conventional indwelling venous cannulas. These can be assigned, e.g. on the basis of a color code, to a defined internal diameter of the venous catheter, or they can be selected by the user depending on the particular application.

The indwelling venous cannula can have at least one holding element for easier application to a living being. Holding elements of this kind can be designed, for example, as projections on the indwelling venous cannula, such that the indwelling venous cannula can be guided with one hand by the user. This makes application easier, since the user can use the other hand, for example, to stabilize the body part of the living being that is to be punctured or to operate an ultrasound appliance.

The indwelling venous cannula can have a fastening element, wherein the fastening element is configured to fix the indwelling venous cannula on the living being. This can have the effect that the indwelling venous cannula cannot accidentally be withdrawn from the punctured body part of the living being. Moreover, the administration of medicaments is also made easier, since the indwelling venous cannula is held fixed on the patient, without being unstable. The indwelling venous cannula can in this case be fixed to the living being by the fastening element, for example, via a self-adhesive wound dressing.

It is conceivable that the fastening element has at least one recess, wherein the recess is configured for guiding a thread. The puncture needle can thus be fixed by being firmly sutured to the living being. By suturing, a tensile force can be applied in the direction of the living being, such that the fixing of the indwelling venous cannula can be improved.

It is also conceivable that the functions of the fastening element and of the holding element are combined in a single element. Thus, in a first step, the user can apply the indwelling venous cannula by means of a combined element of this kind, and, after application, the indwelling venous cannula can be fixed to the patient via the combined element.

It is also conceivable, however, that a click/clip mechanism is used to fix the puncture system to the skin. By means of a click/clip mechanism, fine skin staples or the fastening element are pressed in the direction of the skin, such that the skin staples penetrate into the skin or into the connective tissue of the living being and ensure secure fixing of the indwelling venous cannula. The indwelling venous cannula can then be released at any time, however, by means of the skin staples simply being pulled out of the living being. This procedure can be repeated as often as is desired, if a change of position of the indwelling venous cannula or removal of the latter is necessary.

At the end directed away from the patient, the indwelling venous cannula can have an interposed Y-piece, wherein three-way valves can be mounted on the Y-piece. The three-way valves can in this case be attached to a continuation of the Y-piece. However, it is also conceivable that the three-way valves can be attached directly to the Y-piece. This could be done, for example, with a locking element on the Y-piece, by means of a three-way valve being able to be pushed into a locking element. The Y-piece can be mounted so as to be rotatable about its longitudinal axis, such that the Y-piece can be rotated by the user to an advantageous position. Separate mounting of the three-way valves is also possible, such that a very high degree of flexibility is ensured and optimal operation by the user is permitted. For example, the three-way valves can then be mounted rotatably about their respective axes. Advantageously, a filter can also be integrated in the three-way valves, which filter can prevent, for example, the entry of air, particles or bacteria into the body of a living being.

The indwelling venous cannula can have one or more pressure-sensitive valves which prevent a retrograde flow of liquids and entry of air into the venous catheter and thus into the patient's body. A pressure-sensitive valve of this kind can be designed in such a way that it reacts to a defined differential pressure, e.g. to a pressure difference between arterial and venous pressure. A pressure-sensitives valve of this kind can be designed as a check valve, for example.

The venous catheter of the indwelling venous cannula can have an undulating surface. An undulating surface is distinguished by alternating diameters of the venous catheter at least in cross section along the longitudinal axis of the venous catheter. An undulating shape can consist, for example, of a sinusoidal, rectangular, triangular and/or sawtooth oscillation. It is moreover advantageous if the venous catheter has a spiral-shaped structure and/or is provided with a spiral-shaped structure. By means of the spiral-shaped structure, the venous catheter has great flexibility, which permits easier application to the living being. An indwelling venous cannula is thus made available which is designed to be resistant to cutting and piercing and also flexible.

The venous catheter can additionally be strengthened with further structures or elements, e.g. by transversely, longitudinally or diagonally extending structures or elements, which can also cover or bridge a certain distance of the venous catheter and thus counteract an unspiraling of the venous catheter. These elements can be made of the puncture-resistant material or be designed with a coating of puncture-resistant material.

The venous catheter can be wound, for example, like a spiral spring from a metal, in which case the individual layers can lie tightly on one another, such that they are in contact with one another, similar to the structure of the already known Seldinger wire, which is composed of a tightly wound steel wire. By means of the tightly wound layers, the venous catheter is resistant to cutting and piercing and is at the same time flexible.

Through the density of the windings and through the choice of material of the venous catheter, it is possible to obtain desired properties depending on the particular use, especially as regards the flexibility and stiffness of the venous catheter. Thus, in the case of a tightly wound venous catheter, it is possible to achieve a high degree of stiffness with a high degree of puncture resistance. If the venous catheter is wound less tightly, the flexibility increases. As regards the choice of material, it is also possible to use a combination of different metals or other puncture-resistant materials. It is conceivable that the density of the windings can also be adjusted by the user, if necessary, by means of the wound venous catheter being able to be drawn out or pushed together. It is also possible that the density of the windings is already different at various locations of the venous catheter at the time of manufacture and that the flexibility and stiffness of the venous catheter, particularly in the longitudinal extent, thus already vary at the time of manufacture.

However, it is also conceivable that the venous catheter has grooves distributed about the circumference, e.g. like a corrugated pipe, which grooves provide an undulating surface. A high degree of flexibility of the venous catheter can thus be achieved. The grooves run continuously about the complete circumference of the venous catheter and are advantageously arranged in parallel at regular or irregular intervals along the length of the venous catheter. The venous catheter thus has an alternating diameter. In this way, it is possible that the venous catheter can be oriented in different positions. However, it is also conceivable that the grooves can be distributed in a spiral shape about the circumference. Spiral-shaped grooves of this kind have the advantage that the pressure loss of a fluid conveyed through the venous catheter is reduced and, at the same time, a swirling motion of the fluid can be achieved.

It is advantageous if the undulating surface is provided with a sealing coating, in particular a plastic coating. It is moreover advantageous if the coating is a PTFE coating. The sealing coating can be arranged on the outer face of the venous catheter. Alternatively, the sealing coating can be arranged on the inner face of the venous catheter. It is also possible for the sealing coating to be arranged on the inner face and outer face of the venous catheter in the manner of a sheath. Moreover, the sealing coating can also be let into the undulating surface and can thus seal the undulating surface directly in the region thereof. The sealing coating makes the venous catheter impermeable to gaseous and/or liquid substances. Thus, the venous catheter cannot draw air during aspiration, for example. At the same time, the coating facilitates the insertion of the venous catheter into the punctured body part of the living being.

The venous catheter of the indwelling venous cannula can have, at an end near the patient, a dilation body for uniform widening of a punctured body part. This has the advantage that the venous catheter can be inserted into the punctured body part of the living being without additional widening of the puncture site, since the dilation body over the venous catheter, or a dilation body integrated in the venous catheter, is pushed through the puncture needle into the punctured body part together with the venous catheter, wherein the required dilation takes place and the venous catheter can be pushed farther into the body part.

The dilation body can taper at an acute angle of less than 11 degrees, proceeding from a 360° system. The angle relates to the angular extent between two outer dilation surfaces of the dilation body and not to the center axis of the dilation body.

The dilation body can advantageously taper at an acute angle of less than 10.5 degrees. This has the advantage that the venous catheter can be inserted into the punctured body part easily and without additional widening of the puncture site, by means of the dilation body being pushed over the puncture needle into the punctured body part, wherein the required dilation takes place and the venous catheter can be pushed into the body part. To do this, it is necessary that the dilation body tapers at an acute angle of less than 11 degrees, in particular less than 10.5 degrees, such that an advance through the skin can take place during the dilation procedure. It is conceivable that the dilation body is made of a material, or is coated with a material, that reduces the frictional resistance, so as to make an advance of the dilation body easier. It is also conceivable that, after the dilation body has been inserted into the punctured body part, it is widened and thus widens the puncture site in such a way that the venous catheter can be pushed into the punctured body part.

The venous catheter of the indwelling venous cannula can have, at the end near the patient, recesses which are distributed about the circumference and serve for homogeneous delivery of a fluid into the living being. This has the advantage that the administration of a medicament, for example, into the body of the living being can take place more homogeneously than in the case of conventional indwelling cannulas, such that the medicament is not undesirably concentrated at one location. Moreover, the sampling of blood, for example, or of other liquids is improved, similarly also the drainage of secretions or of air, for example, depending on the particular use.

In contrast to conventional indwelling cannulas, the venous catheter of the indwelling venous cannula according to the invention can be designed not just with one lumen but also with at least two separate lumens. The puncture needle is guided through one of the lumens. A multi-lumen indwelling venous cannula is thus made available through which medicaments and solutions can be simultaneously administered separately from one another. By means of recesses or outlet holes arranged laterally on the venous catheter, mixing of different medicaments and solutions can be avoided. By arranging a plurality of lateral recesses or outlet holes, the flow rates of the administered liquids can also be increased. Pressure infusions can advantageously be used. The structure of the above-described puncture-resistant material can also be adapted here to the presence of the lateral outlet holes, e.g. in a ring shape around the outlet holes, in order to additionally stabilize these or keep them open.

In the multi-lumen design of the indwelling venous cannula, it is possible that the recesses/outlet holes are also arranged at the top and bottom of the venous catheter. In a multi-lumen design of the indwelling venous cannula, it is also advantageous to have a plurality of attachment elements for attaching an aspiration element. This can permit improved monitoring of the position of the indwelling venous cannula or of the venous catheter in a blood vessel, for example, since the different portions of the venous catheter can be monitored in terms of their position in a blood vessel, for example. This has medical relevance, for example, in the puncturing of deep-lying blood vessels. If the lumen for the return flow of blood is monitored, it is generally possible to assume that all the other lumens also end in the blood vessel, since their openings are arranged still deeper in the blood vessel. The different openings can also each be lined with or surrounded by an X-ray visible material for precise monitoring of the position of the openings and thus also for exact monitoring of the position of the indwelling venous cannula or of the venous catheter. The attachment element or the attachment elements can be arranged on the indwelling venous cannula in such a way that they point away from the patient's skin at a certain angle, for example at a 90 degree angle or a 45 degree angle.

In one embodiment of the indwelling venous cannula in the multi-lumen design, it is possible, for example, that the puncture-resistant material is present only in that part of the venous catheter in which the puncture needle is guided in a longitudinally displaceable manner.

Over the entire length or at defined locations, the indwelling venous cannula or the venous catheter can contain or be coated with a material which is visible by X-ray and thus permits exact monitoring of the position of the indwelling venous cannula or of the venous catheter in the body.

The venous catheter of the indwelling venous cannula can have an attachment element for attaching an aspiration element, e.g. a syringe. It is thus possible at any time to perform aspiration in order to monitor whether the venous catheter is located in the target structure of the living being. By aspiration of blood, for example, it is possible to verify whether the venous catheter of the indwelling venous cannula is still located in a blood vessel. Furthermore, an attachment element of this kind permits simplified administration of a medicament.

The puncture needle of the indwelling venous cannula is advantageously a hollow needle. A hollow needle allows the user, during and after the puncturing procedure, to aspirate blood in order to ascertain, directly during and after the puncturing procedure, whether the corresponding vessel has already been correctly punctured.

An expansion body for fixing the position of the venous catheter in the punctured body part can be arranged on the venous catheter. An expansion body of this kind can be designed, for example, as an inflatable cuff, which is arranged for example on the outer face of the venous catheter. With such a cuff, the indwelling venous cannula can be sealed off from the outside by means of the cuff, introduced under the skin, being inflated or filled with liquid, whereby the puncture system is thus sealed off from the outside and also advantageously fixed in its position.

The indwelling venous cannula can have a hollow extension element, wherein a fluid is able to flow through the hollow extension element in order to reduce clot formation. When a fluid, e.g. a saline solution, flows through the extension element and therefore the venous catheter, this prevents a situation where clots form in the venous catheter and make use of the indwelling venous cannula impossible.

However, it is also conceivable that a mandrel is used which is arranged inside the venous catheter such that formation of clots is prevented.

The indwelling venous cannula can have a safety mechanism which is configured to shield the tip of the puncture needle after the puncture needle has been removed from the venous catheter. Such shielding prevents a situation where the user or the living being is injured by the tip of the puncture needle when the latter is withdrawn from the indwelling venous cannula after the puncture procedure has been completed.

The puncture needle of the indwelling venous cannula can be enclosed by a protective cap which is removed before the indwelling venous cannula is used. Injuries during the handling of the indwelling venous cannula can thus be avoided.

By means of the indwelling venous cannula, a local anesthetic and/or antimicrobial substances can be introduced into the body of the patient continuously or at intervals. It is thus possible, for example, to reduce pain during the insertion or placement of the indwelling venous cannula.

The indwelling venous cannula can have a needlestick guard for the puncture needle. At least when the indwelling venous cannula is new, the needlestick guard ensures that the user cannot injure himself on the tip of the puncture needle.

Depth markings can be arranged on the venous catheter. The depth markings allow the user to monitor the depth of insertion of the catheter in the patient. The venous catheter can additionally have a stop ring mounted thereon in order to limit the depth of insertion of the catheter to a defined extent.

The invention is explained in more detail below on the basis of an illustrative embodiment and with reference to a drawing, in which:

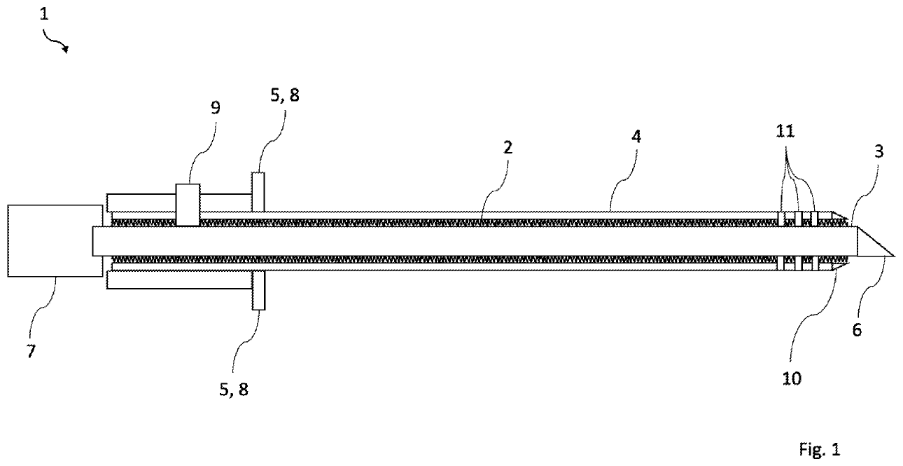

FIG. 1 shows a schematic side view of an indwelling venous cannula.

FIG. 1 shows a schematic side view of an indwelling venous cannula 1. The indwelling venous cannula 1 has a venous catheter 2, wherein a puncture needle 3 can be guided in a longitudinally displaceable manner in the venous catheter 2. The indwelling venous cannula 1 is designed here as a peripheral indwelling venous cannula 1. It becomes clear that the venous catheter 2 has been formed as a tightly wound spiral spring from a puncture-resistant material, such that an undulating surface is obtained. The venous catheter 2 is here made of a puncture-resistant material over its entire length. The flexibility of the venous catheter 2 is ensured by the spiral-shaped structure. Such a design of the venous catheter 2 provides piercing and cutting protection, which protects the venous catheter 2 from being pierced through, for example, by a patient-side tip 6 of the puncture needle 3 during the application of the indwelling venous cannula 1. This minimizes the danger of damaging the venous catheter 2. The latter can no longer be sheared off by the patient-side tip 6 of the puncture needle 3, even after repeated displacement of the puncture needle 3 relative to the venous catheter 2. The quantity of rejects resulting from damaged venous catheters can thus be greatly reduced. Moreover, repeat use of the indwelling venous cannula 1 in the context of a puncturing procedure is possible under permanently sterile conditions on a living being. This is of importance particularly if an inaccurate puncture was initially made, that is to say the blood vessel was accidentally missed in the first puncture, or the venous catheter 2 was unable to be pushed sufficiently far into the blood vessel at the first attempt.

For improved aspiration, the venous catheter 2 is provided with a sealing coating 4. The undulating surface can result in very small openings being created, which make aspiration difficult, for example because air can be drawn undesirably through these openings. A sealing coating 4 can minimize or prevent the undesired aspiration of air. The sealing coating 4 is advantageously a PTFE coating, which at the same time facilitates the insertion of the venous catheter 2 into the punctured body part. By means of an additional dilation element 10 at the patient-side end of the venous catheter 2, a uniform widening is achieved as the venous catheter 2 is advanced into the punctured body part.

The application of the indwelling venous cannula 1 to a living being can proceed with the following steps, for example:
1. Puncturing a vein with the puncture needle 3.
2. Advancing the spiral-shaped venous catheter 2, with the sealing coating 4, past the patient-side tip 6 of the puncture needle 3 into the vein as far as the desired position.
3. Removing/retracting the puncture needle 3.
4. Closing the spiral-shaped venous catheter 2 at an end remote from the patient.

The indwelling venous cannula 1 has two holding elements 5. These holding elements 5 allow the user to operate the indwelling venous cannula 1 with one hand, while the second hand can be used, for example, to stabilize the body part that is to be punctured. The puncture needle 3 is designed as a hollow needle. After the puncture has been made by the patient-side tip 6 of the puncture needle 3, the user can immediately ascertain whether the vein has been correctly punctured, since the hollow puncture needle 3 fills with venous blood and passes into the chamber 7, as a result of which the blood can be immediately observed by the user.

After the puncture has been made, the venous catheter 2 can be pushed into the punctured body part and, at the same time, the puncture needle 3 and the chamber 7 are withdrawn from the components of the indwelling venous cannula 1 that remain in the body part. A safety mechanism can be designed such that the patient-side tip 6 of the puncture needle 3 is shielded after withdrawal from the indwelling venous cannula 1, thereby protecting the user and the living being from possible needlestick injuries.

In its end position in the punctured body part, the venous catheter 2 can be held fixed on the living being via fastening elements 8. The fixing can be effected using a self-adhesive wound dressing, which fixes the indwelling venous cannula 1 on the living being via the fastening elements 8. The described fastening elements 8, which can be designed as wings for example, are optional elements of the indwelling venous cannula 1.

The puncture needle can in this case extend substantially in the center between the holding elements 5 and/or the fastening elements 8.

It is clear that the functions of the holding element 5 and of the fastening element 8 can be combined in one element. This permits simple production of the indwelling venous cannula 1, while at the same time its design makes the indwelling venous cannula 1 easy for the user to hold.

An aspiration element, for example a syringe, can be attached via an attachment element 9. The attachment element 9 can be configured here as a valve which permits simple administration of a medicament or an aspiration of blood. The valve in the free state prevents a retrograde flow of liquids, e.g. blood, out of the attachment element 9. Moreover, the valve in the free state prevents entry of air from the outside into the attachment element 9. The attachment element 9 can moreover comprise a filter which prevents entry of large particles, bacteria and air into the interior of the attachment element 9 and thus into the interior of the indwelling venous cannula.

An aspiration element, for example a syringe, can be attached to the chamber 7. The indwelling venous cannula can in this way be inserted into a vein under continuous aspiration with the syringe. The outcome of the puncture can thus be ascertained directly and with great precision. The chamber 7 can also be designed here as a further valve, which permits the flow of fluids in only one defined direction. Alternatively or additionally, the chamber 7 can also be designed such that it prevents the entry of air or allows air and other gases and vapors to pass through in only one defined direction. The chamber 7 can be designed, for example, like the attachment element 9.

The chamber 7 and the attachment element 9 can be covered by a protective cap, such that undesired contamination does not take place when the chamber 7 and the attachment element 9 are not being used. The protective cap can be connected by a respective tab to the chamber 7 and/or to the attachment element 9.

At the end near the patient, the venous catheter 2 has recesses 11 distributed about the circumference. The recesses 11 permit homogeneous dispensing of a medicament, for example, into the living being. It is thereby possible to avoid the medicament being dispensed locally at an undesirably high concentration. By the provision of a plurality of recesses 11, it is also possible to increase the flow rates of the administered infusion solutions and medicaments. Moreover, it is thus also easier to aspirate liquids, e.g. to collect blood, from the living being via the applied venous catheter or via the applied indwelling venous cannula. With a suitable design of the other components of the indwelling venous cannula, a desired retrograde spontaneous exit of fluids, vapors and/or gases from the indwelling venous cannula is also possible when the indwelling venous cannula is intended for use for draining fluids, vapors and/or gases, for example in the context of puncturing the pleural space, other cavities or the above-described interstices.

The present illustration is only a schematic illustration that provides a good overview of the components of the indwelling venous cannula according to the invention. The lengths and size ratios may differ, however, in reality.

FIG. 1 is to be understood as a possible illustrative embodiment. Other forms of the teaching according to the invention are also conceivable. Furthermore, the elements of the illustrative embodiment are not linked inseparably to one another, and therefore, for example, the implementation of the invention is not dependent on the specifically described elements of the illustrative embodiment. It is thus always conceivable, for example, for there to be variability in the number, length or size of the individual elements.

### LIST OF REFERENCE SIGNS

1—indwelling venous cannula

2—venous catheter

3—puncture needle

4—sealing coating

5—holding element

6—tip

7—chamber

8—fastening element

9—attachment element

10—dilation body

11—recesses

1. An indwelling venous cannula for application to a living being, comprising a venous catheter, wherein a puncture needle can be guided in a longitudinally displaceable manner in the venous catheter, characterized in that at least the part of the venous catheter designed to remain in the living being is formed from a puncture-resistant material, or is coated with such a puncture-resistant material, over its entire length or most of its length in which the puncture needle can be guided in a longitudinally displaceable manner.

2. The indwelling venous cannula as claimed in claim 1, wherein the indwelling venous cannula is a peripheral indwelling venous cannula.

3. The indwelling venous cannula as claimed in claim 2, wherein the venous catheter of the peripheral indwelling venous cannula has a piercing length of 10 mm to 200 mm.

4. The indwelling venous cannula as claimed in claim 1, wherein the indwelling venous cannula has at least one holding element for easier application to the living being.

5. The indwelling venous cannula as claimed in claim 1, wherein the indwelling venous cannula has a fastening element, wherein the fastening element is configured to fix the indwelling venous cannula on the living being.

6. The indwelling venous cannula as claimed in claim 1, wherein the venous catheter has an undulating surface.

7. The indwelling venous cannula as claimed in claim 6, wherein the venous catheter has a spiral-shaped structure and/or is provided with a spiral-shaped structure.

8. The indwelling venous cannula as claimed in claim 6, wherein the undulating surface is provided with a sealing coating, in particular a plastic coating.

9. The indwelling venous cannula as claimed in claim 8, wherein the sealing coating is a PTFE coating.

10. The indwelling venous cannula as claimed in claim 1, wherein the venous catheter has, at an end near the patient, a dilation body for uniform widening of a punctured body part.

11. The indwelling venous cannula as claimed in claim 1, wherein the venous catheter has, at the end near the patient, recesses which are distributed about the circumference and serve for homogeneous delivery of a fluid into the living being.

12. The indwelling venous cannula as claimed claim 1, wherein the venous catheter has an attachment element for attaching an aspiration element.

13. The indwelling venous cannula as claimed in claim 1, wherein the puncture needle is a hollow needle.

14. The indwelling venous cannula (1) as claimed in claim 1, wherein an expansion body for fixing the position of the venous catheter in the punctured body part is arranged on the venous catheter.

15. The indwelling venous cannula as claimed in claim 1, wherein the indwelling venous cannula has a hollow extension element, wherein a fluid is able to flow through the hollow extension element in order to reduce clot formation.

16. The indwelling venous cannula as claimed in claim 1, wherein the indwelling venous cannula has a safety mechanism which is configured to shield the tip of the puncture needle after the puncture needle has been removed from the venous catheter.
